(19) United States
(12) Patent Application Publication
(10) Pub. No.: US 2026/0306000 A1
(43) Pub. Date: Oct. 8, 2026

(54) INTERFACE STRUCTURE FOR A BIOELECTRODE

(71) Applicant: TEKNOLOGIAN TUTKIMUSKESKUS VTT OY, Espoo (FI)

(72) Inventors: Teemu RUOTSALAINEN, Espoo (FI); Tomi MATTILA, Espoo (FI); Juha LEUKKUNEN, Espoo (FI)

(21) Appl. No.: 19/482,054

(22) PCT Filed: May 8, 2024
(86) PCT No.: PCT/FI2024/050220

§ 371 Date: Nov. 6, 2025

(30) Foreign Application Priority Data
May 10, 2023 (FI) 20235527

(51) Int. Cl. A61B 5/263; A61B 5/259; A61K 31/015; A61K 31/07; A61K 31/122; A61K 31/355; A61K 31/375; A61K 33/04; A61K 33/26; A61K 33/30; A61K 33/34; A61K 36/886; A61K 45/06

(52) U.S. Cl. CPC A61B 5/263; A61B 5/259; A61K 31/015; A61K 31/07; A61K 31/122; A61K 31/355; A61K 31/375; A61K 33/04; A61K 33/26; A61K 33/30; A61K 33/34; A61K 36/886; A61K 45/06

(57) ABSTRACT

A bioelectrode including an electrode structure with a carrier, a conductive element, and an adhesive layer which forms an outermost surface of the electrode structure; and a skin interface structure on the outermost surface of the electrode structure, including an electrically conductive film with at least one opening in it. The advantage using the skin interface structure with the electrode structure is that the quality of the measured signal may be improved.

### FIELD OF THE DISCLOSURE

The disclosure relates to a bioelectrode comprising an interface structure.

### BACKGROUND OF THE DISCLOSURE

Biopotential measurements such as electrocardiogram (ECG) or electromyogram (EMG) are popular tests in cardiology and neurology. Signal quality in biopotential measurements is a critical factor for correct interpretation and effective disease diagnostics. One of the greatest problems in biopotential measurements is motion artifacts that arise when the patient is moving. The motion artifacts make data analysis more complicated which may delay diagnosis or cause misdiagnosis.

One way to improve the quality of measured signal is by using signal post-processing applied to the raw signal. However, software-based processing of the raw data of poor quality requires extra time and effort. Specifically, the analysis of 24 h Holter data measurement may require 15 minutes with high quality raw data and up to 3 days with low quality of raw data.

Alternative way to eliminate motion artifacts is to develop new types of bioelectrodes. KR101668022B1 discloses a polymer electrode with an adhesive layer and a support layer and demonstrates that the adhesive layer has an effect of preventing operation noise by closely adhering the polymer electrode the moving body. However, producing new type of electrode and introducing it into wide use is expensive and time consuming. Thus, a solution which can be combined with the widely available commercial electrodes and improve their signal quality is highly desired.

### BRIEF DESCRIPTION OF THE DISCLOSURE

An object of the present disclosure is to provide an improvement to the design of the bioelectrodes so as to solve the above problem.

The object of the disclosure is achieved by what is stated in the independent claim. The preferred embodiments of the disclosure are disclosed in the dependent claims.

The disclosure is based on the idea of using an interface structure between an outermost surface of an electrode structure and the skin during biosignal measurement. An advantage of arrangement of the disclosure is that it improves raw signal quality by alleviating motion artefacts and reducing contact impedance. Additionally, it is easy to fabricate, inexpensive, and can be combined with wide range of existing commercial electrodes.

### BRIEF DESCRIPTION OF THE DRAWINGS

In the following the disclosure will be described in greater detail by means of preferred embodiments with reference to the accompanying drawings, in which

FIG. 1 illustrates a) an interface structure and an electrode structure intended to be attached to each other; b) a schematic of the skin interface structure attached to an outermost surface of the electrode structure; and c) a photo of a bioelectrode with the skin interface structure attached to the outermost surface of the electrode structure.

FIG. 2a illustrates one example of the electrode structure (cross-section) with which the skin interface structure may be used.

FIG. 2b illustrates the skin interface structure attached to the electrode structure (cross-section).

FIG. 2c illustrates an embodiment of this disclosure comprising the skin interface structure attached to the electrode structure (cross-section). FIG. 2d illustrates the electrode structure (cross-section) with the adhesive layer comprising a skin caring layer.

FIG. 2e illustrates the bioelectrode (cross-section) with the skin interface structure comprising the skin caring layer.

FIG. 3a illustrates different embodiments of the skin interface structure: examples a)-c) comprise supporting structure, and examples d)-f) have no supporting structure. The embodiments also demonstrate different geometries of the skin interface structure.

FIG. 3b is a photograph of the embodiment comprising the electrode structure with the skin interface structure.

FIG. 4a illustrates an exemplary ECG measurement setup.

FIG. 4b illustrates the data obtained during the measurement with and without the skin interface structure a) 30 minutes after attachment to the skin and b) 1 hour after the first measurement.

### DETAILED DESCRIPTION OF THE DISCLOSURE

A bioelectrode comprising an electrode structure comprising a carrier with a top surface and a bottom surface, a conductive element extending through the carrier to the bottom surface, an adhesive layer which at least partly covers the bottom surface of the carrier and forms an outermost surface of the electrode structure; characterized in that the bioelectrode further comprises a skin interface structure on the outermost layer of the electrode structure, and the skin interface structure comprises an electrically conductive film with at least one opening in it.

### Definition of Bioelectrode

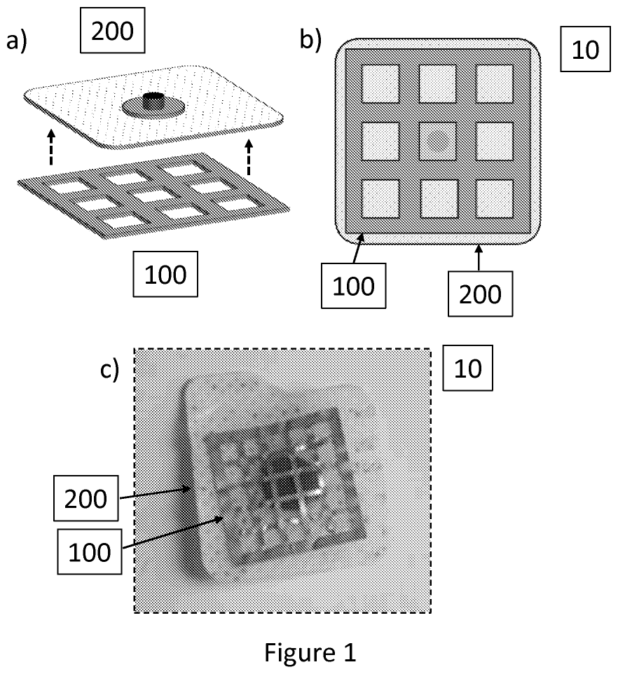

A bioelectrode 10 of this disclosure comprises a combination of an electrode structure 200 and a skin interface structure 100 as illustrated in FIG. 1 a)-c). The skin interface structure 100 may be configured to attach to an outermost surface of the electrode structure 200. The details of each structure and the ways of combining them are discussed further.

### Electrode Structure Definition

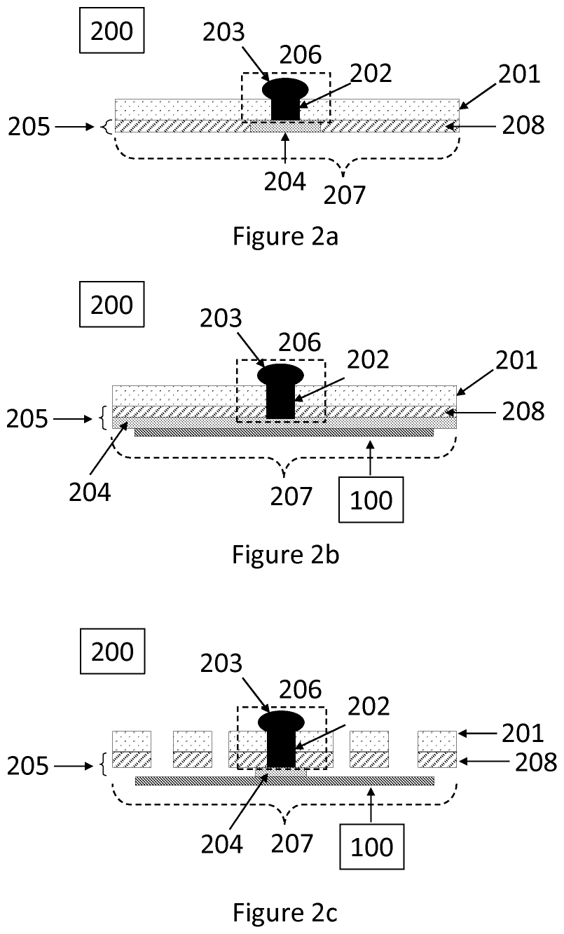

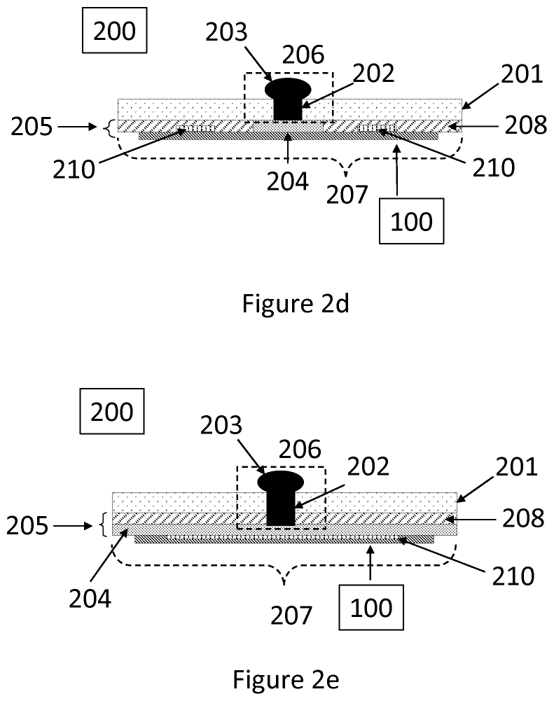

The electrode structure 200 may comprise the following components illustrated in FIGS. 2a-c:
A carrier 201 which comprises a top surface and a bottom surface;
A conductive element 206 extending through the carrier to the bottom surface, and the conductive element 206 may comprise:
   A conductive core 202 which may be extending from the top to the bottom surface of the carrier. The conductive core 202 may be a metallic pin. The conductive core 202 may extend beyond the bottom surface of the carrier 201. Optionally, a connector snap 203 at the top surface of the carrier connected to the conductive core 202. The connector snap 203 may be connected to the measurement cables (not illustrated).

The conductive element 206 may alternatively extend through the carrier 201 from the top surface to the bottom surface. Other options of how the conductive element 206 may be integrated into the carrier 201 are possible. The conductive element 206 may be electrically conductive.
An adhesive layer 205 at the bottom surface of the carrier, which helps to secure the electrode in place during monitoring. The adhesive layer may cover 10-100% of the bottom surface of the carrier. The adhesive layer may cover more than 10%, more than 25%, more than 50%, more than 75% of the bottom surface of the carrier. The adhesive layer may cover less than 100%, less than 65%, less than 40%, less than 15% of the bottom surface of the carrier. The adhesive layer may comprise at least one of the following components:
   A carrier adhesive 208 may be deposited at the bottom surface of the carrier. The carrier adhesive 208 helps to improve the adhesion between the electrode structure and the skin and reduce its movement of the electrode. The carrier adhesive 208 may cover the entire bottom surface of the carrier around the conductive core 202. It may alternatively cover part of the bottom surface of the carrier. Alternatively, the conductive core 202 may extend through the carrier and the carrier adhesive 208. In that case, the carrier adhesive 208 may comprise opening in it. The carrier adhesive 208 may be electrically non-conductive. Alternatively, the carrier adhesive 208 may be electrically conductive and called a conductive carrier adhesive. In that case, the carrier adhesive 208 may also cover the conductive core 202.
   A conductive layer 204 may be applied at the bottom surface of the carrier 201 in contact with the conductive core 202. The conductive layer 204 may be electrically conductive. The conductive layer 204 helps to improve the electrode core's conductivity and reduce interference which may arise from the movement of the electrode on the skin. The conductive layer 204 may partly cover the bottom surface of the carrier 201 as illustrated in FIG. 2a. Alternatively, the conductive layer 204 may cover the entire bottom surface of the carrier 201 (not illustrated), or the entire surface of the carrier adhesive 208 as in FIG. 2b. Alternatively, the conductive layer 204 may cover part of the carrier adhesive 208 as in FIG. 2c. The conductive layer 204 may be metal. The conductive layer may be adhesive. Thus, the conductive layer may be called a conductive adhesive.

The carrier adhesive 208 and the conductive layer 204 may be combined to form at least part of the adhesive layer 205. In other words, the adhesive layer may comprise the carrier adhesive 208 and the conductive layer 204.
The conductive layer 204 may cover part of the bottom surface of the carrier 201 and form contact with the conductive core 202 as in FIG. 2a. The carrier adhesive 208 may cover another part of the bottom surface of the carrier 201 around the conductive layer 204. In this case, both carrier adhesive 208 and the conductive layer 204 may be configured to be in contact with the skin.
The carrier adhesive 208 may cover at least part of the bottom surface of the carrier 201 around the conductive core 202 as in FIG. 2b. The conductive layer 204 may cover the entire carrier adhesive 208. In this case, the conductive layer may be adhesive and may be configured to adhere to the skin.

The adhesive layer 205 may further comprise other layers of elements which are not presented in this disclosure. Importantly, the electrode structure 200 may also comprise other elements and parts not described above or below.

The electrode structure 200 may comprise the outermost surface 207. The outermost surface 207 may be defined as the surface of the electrode structure 200 which is configured to be in contact with the skin. Depending on the electrode structure, the outermost surface 207 may be at least partly formed by the adhesive layer 205. In other words, a combination of the carrier adhesive 208 and the conductive layer 204 may form at least part of the outermost surface 207 of the electrode structure 200, as in FIGS. 2a and 2c. Alternatively, only the conductive layer 204 may form the outermost surface 207 of the electrode structure 200, as in FIG. 2b. The at least part of the outermost surface 207 of the electrode structure 200 may be adhesive. Further, the at least part of the outermost surface 207 of the electrode structure 200 may be electrically conductive.

FIG. 2b illustrates the electrode structure 200 with the skin interface structure 100 attached to its outermost surface 207. In this example, the outermost surface 207 may be formed by the conductive layer 204. The skin interface structure 100 may be used with other electrode structure different from the one illustrated in FIG. 2. The described example should not be viewed in a limiting sense.

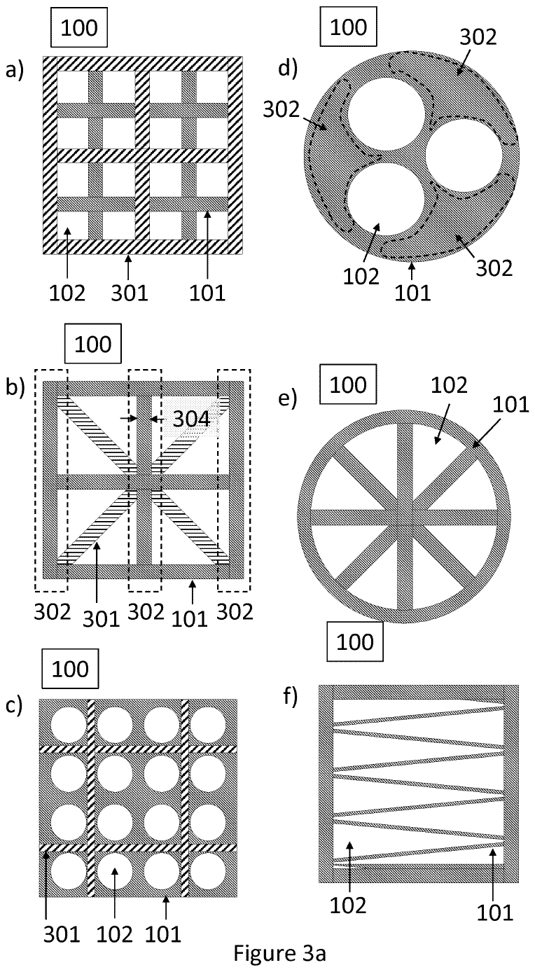

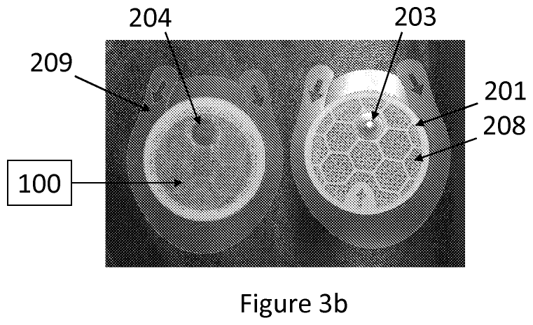

The carrier 201 of the electrode structure 100 may comprise at least one opening in it. The carrier adhesive 208 may also comprise at least one opening in it. The carrier 201 and the carrier adhesive 208 may comprise at least one opening different from the opening through which the conductive element extends. The carrier 201 and the carrier adhesive 208 with openings are illustrated in FIGS. 2c and 3b. If present in both the carrier 201 and the carrier adhesive 208, the density of the openings of the carrier 201 may be smaller than the density of the openings of the carrier adhesive 208, as in FIG. 3b. If present in both the carrier 201 and the carrier adhesive 208, the size of the openings of the carrier 201 may be larger than the density of the openings of the carrier adhesive 208. Alternatively, if present in both the carrier 201 and the carrier adhesive 208, the density and the size of the openings of the carrier 201 may be equal to the density and the size of the openings of the carrier adhesive 208, as in FIG. 2c. Specifically, the openings may occupy 1-99.9 % of the carrier 201. The openings may occupy more than 1%, more than 20%, more than 40%, more than 60%, more than 80% of the carrier 201. The openings may occupy less than 99.9%, less than 90%, less than 70%, less than 50%, less than 30%, less than 10% of the carrier 201. Specifically, the openings may occupy 1-99.9 % of the carrier adhesive 208. The openings may occupy more than 1%, more than 20%, more than 40%, more than 60%, more than 80% of the carrier adhesive 208. The openings may occupy less than 99.9%, less than 90%, less than 70%, less than 50%, less than 30%, less than 10% of the carrier adhesive 208.

In total, the carrier 201 may comprise 1-3000000 openings. In total, the carrier adhesive 208 may comprise 1-3000000 openings. The carrier 201 and the carrier adhesive 208 may each comprise more than 1, more than 2, more than 10, more than 100, more than 500, more than 1000, more than 5000, more than 50000, more than 100000, more than 1000000 openings. The carrier 201 and the carrier adhesive 208 may each comprise less than 3000000, less than 1500000, less than 75000, less than 20000, less than 10000, less than 8000, less than 2500, less than 750, less than 150, less than 75 openings.

The openings may extend from the top surface to the bottom surface of each the carrier 201 and the carrier adhesive 208. The openings of the carrier 201 may have a diameter of 0.2 um-5 mm. The openings of the carrier adhesive 208 may have a diameter of 0.2 um-5 mm. The openings of the carrier 201 and the carrier adhesive 208 may have a diameter of more than 0.2 um, more than 0.5 um, more than 1 um, more than 5 um, more than 20 um, more than 50 um, more than 100 um, more than 250 um, more than 500 um, more than 1 mm, more than 3 mm, more than 4,5 mm. The openings of the carrier 201 and the carrier adhesive 208 may have a diameter of less than 5 mm, less than 2.5 mm, less than 800 um, less than 450 um, less than 200 um, less than 75 um, less than 35 um, less than 10 um, less than 4 um, less than 0.8 um, less than 0.4 um. The openings of the carrier 201 and the carrier adhesive 208 may have different diameters. Alternatively, the openings of the carrier 201 and the carrier adhesive 208 may have same diameters.

The openings of each the carrier 201 and the carrier adhesive 208 may have circular shape. Alternatively, the openings of each the carrier 201 and the carrier adhesive 208 may have rectangular shape. Alternatively, the openings of each the carrier 201 and the carrier adhesive 208 may have hexagonal shape. The openings may extend from the top surface to the bottom surface of each the carrier 201 and the carrier adhesive 208. The openings may be straight channels which extend from the top surface to the bottom surface of each the carrier 201 and the carrier adhesive 208. Alternatively, the openings may be curved. Alternatively, the openings may extend horizontally from one side to the other side of each the carrier 201 and the carrier adhesive 208. Alternatively, the openings may extend both from the top surface to the bottom surface of each the carrier 201 and the carrier adhesive 208 and horizontally from one side to the other side of each the carrier 201 and the carrier adhesive 208. The purpose of the openings in the carrier 201 and the carrier adhesive 208 may be to provide air passing through the electrode structure 200 towards the skin when it is in contact with the skin. In addition, the openings may be used as evaporation channels to let moisture pass from the skin to the air.

The shape and the size of the electrode structure 200 may be defined by the size and shape of the carrier. The electrode structure 200 may have a rectangular shape. The length of the edge of the rectangular electrode structure 200 may be 10-60 mm. For example, neonatal size of the rectangular electrode structure 200 may be 15×22 mm. The adult size of the rectangular electrode structure 200 may be 43×50 mm. The length of the edge of the rectangular electrode structure 200 may be more than 10 mm, more than 15 mm, more than 25 mm, more than 35 mm, more than 45 mm, more than 55 mm. The length of the edge of the rectangular electrode structure 200 may be less than 60 mm, less than 50 mm, less than 40 mm, less than 30 mm, less than 20 mm, less than 12 mm. The electrode structure 200 may have a circular shape. The diameter of the circular electrode structure 200 may be 10-70 mm. For example, neonatal diameter of the circular electrode structure 200 may be 12 mm. The adult diameter of the circular electrode structure 200 may be 55 mm. The diameter of the circular electrode structure 200 may be more than 10 mm, more than 15 mm, more than 25 mm, more than 35 mm, more than 45 mm, more than 55 mm, more than 65 mm. The diameter of the circular electrode structure 200 may be less than 70 mm, less than 60 mm, less than 50 mm, less than 40 mm, less than 30 mm, less than 20 mm, less than 12 mm.

### Definition of the Skin Interface Structure

The skin interface structure 100 of this disclosure may comprise a thin sheet of electrically conductive material 101 with at least one opening 102 in it as illustrated in FIG. 3a. The at least one opening of the skin interface structure 100 may alternatively be called a perforation. The thin sheet of electrically conductive material with at least one opening may also be called a mesh 101 or a conductive mesh 101. Further, the skin interface structure 100 may comprise additional elements integrated or attached to the conductive mesh. The skin interface structure 100 may have a top surface and a bottom surface. The openings may extend from the top surface to the bottom surface of the skin interface structure 100. The openings may be straight channels which extend from the top surface to the bottom surface of the skin interface structure 100. Alternatively, the openings may be curved channels. The skin interface structure 100 may be configured to attach to an outermost surface 207 of the electrode structure 200 with its top surface as illustrated in FIG. 1 a). The skin interface structure 100 may be configured to attach to the skin with its bottom surface. In other words, the skin interface structure 100 may be between the skin and the outermost surface 207 of the electrode structure 200.

### Physical Properties of the Conductive Mesh

The mesh 101 may comprise more than one opening 102. The openings may occupy 1-99.6 % of the skin interface structure 100. Specifically, the openings may occupy more than 1%, more than 5%, more that 10%, more than 20%, more than 40%, more than 50%, more than 65%, more than 80%, more than 90% of the skin interface structure 100. The openings may occupy less than 99.6%, less than 85%, less than 75%, less than 55%, less than 35%, less than 25%, less than 12%, less than 7% of the skin interface structure 100. Alternatively, the mesh 101 may comprise axially 1-200 openings per inch. The mesh 101 may have more than 1, more than 5, more than 10, more than 50, more than 100, more than 150 openings per inch. The mesh 101 may have less than 200, less than 120, less than 80, less than 40, less than 15, less than 7 openings per inch. In total, the mesh 101 may comprise 1-40000 openings. Specifically, the mesh 101 may comprise more than 1, more than 10, more than 50, more than 100, more than 500, more than 1000, more than 5000, more than 15000, more than 25000, more than 35000 openings. The mesh 101 may comprise less than 40000, less than 30000, less than 20000, less than 10000, less than 2500, less than 700, less than 250, less than 75, less than 25, less than 5 openings.

The mesh 101 may have rectangular shape as in FIGS. 1 and 3 a)-c). A length of the edge of the rectangular mesh may be 5-50 mm. The lengths of all edges may be equal. The lengths of the edges may be alternatively different. Specifically, the length of the edge of the rectangular mesh may be more than 5 mm, more than 10 mm, more than 20 mm, more than 30 mm, more than 40 mm. The length of the edge of the rectangular mesh may be less than 50 mm, less than 35 mm, less than 25 mm.

Alternatively, the mesh 101 may have circular shape as in FIG. 3 a d). A diameter of the circular mesh may be 5-70 mm. Specifically, the diameter of the circular mesh may be more than 5 mm, more than 10 mm, more than 20 mm, more than 40 mm, more than 45 mm, more than 55 mm, more than 65 mm. The diameter of the circular mesh may be less than 70 mm, less than 60 mm, less than 50 mm, less than 35 mm, less than 25 mm. Alternatively, the mesh may have other shapes such as triangular or spiral shape. The dimensions of the mesh of other shapes may correspond to the dimensions of the mesh of circular or rectangular shapes.

The mesh may be described as a sheet of material having one or more openings. The material around the openings may be shaped into interconnected mesh portions. Alternatively, the mesh may be described as the plurality of intercrossed threads or wires which form the mesh portions. The examples of mesh portions 302 are shown in FIG. 3a. The mesh portions 302 may be equal in width and length through the entire mesh as in FIG. 3a b). Alternatively, the mesh portions may be equal in area as in FIG. 3a d). However, the mesh portion may be different in width, length or area depending on the distribution of the openings in the sheet of material.

The openings 102 may have circular shape. A diameter of the circular openings may be in the range of 0.2-50 mm. Alternatively, the openings may have rectangular shape. A length of the edge of the rectangular openings may be in the range of 0.2-50 mm. The diameter of the circular openings and the length of the edge of the rectangular openings may be more than 0.2 mm, more than 0.5 mm, more than 1 mm, more than 3 mm, more than 5 mm, more than 7 mm, more than 10 mm, more than 20 mm, more than 30 mm, more than 40 mm. The diameter of the circular openings and the length of the edge of the rectangular openings may be less than 50 mm, less than 45 mm, less than 35 mm, less than 25 mm, less than 15 mm, less than 9 mm, less than 6 mm, less than 2 mm, less than 1.5 mm, less than 0.4 mm. Alternatively, the diameter of the circular openings and the length of the edge of the rectangular openings may be 0.2 um-50 mm. The diameter of the circular openings and the length of the edge of the rectangular openings may be less than 0.2 mm, less than 0.1 mm, less than 50 um, less than 10 um, less than 5 um, less than 1 um, less than 0.5 um. The diameter of the circular openings and the length of the edge of the rectangular openings may be less more than 0.2 um, more than 0.8 um, more than 2 um, more than 8 um, more than 25 um, more than 75 um, more than 0.15 mm. Alternatively, the openings may have other shapes such as tringles or spiral shape. The dimensions of the openings of other shape may correspond to the dimensions of the circular and rectangular openings listed above.

The size and the number of the openings may define the size and the number of mesh portions 302. For example, a width 304 of the mesh portions 302 of the rectangular mesh with rectangular openings as in FIG. 3 a b) may be 0.05-2 mm. The width 304 of the mesh portions 302 may be more than 0.05 mm, more than 0.1 mm, more than 0.5, more than 1 mm, more than 1.5 mm. The width of the mesh portion may be less than 2 mm, less than 1.7 mm, less than 1.2 mm, less than 0.7 mm, less than 0.3 mm, less than 0.15 mm. An area of the mesh portions 302 may be 0.04-2500 mm². The area of the mesh portions 302 may be more than 0.04 mm², more than 1 mm², more than 5 mm², more than 50 mm², more than 100 mm², more than 500 mm², more than 1000 mm², more than 1500 mm², more than 2000 mm². The area of the mesh portions 302 may be less than 2500 mm², less than 1800 mm², less than 1200 mm², less than 800 mm², less than 400 mm², less than 200 mm², less than 70 mm², less than 20 mm², less than 7 mm², less than 2 mm², less than 0.07 mm².

The same range of parameters of the openings of the skin interface structure 100 defined above may apply to the optional openings in the carrier 201 and the carrier adhesive 208.

### Materials of the Conductive Mesh

The material of at least part of the conductive film of this disclosure may be metal. A thickness of such conductive film may be in the range of 10nm-50 um.

The material of the mesh 101 may be electrically conductive metal. Specifically, the material of the mesh may be copper, gold, silver, stainless steel and so on. A thickness of metal mesh may be in the range of 10 nm-50 um. Specifically, the thickness of metal mesh may be more than 10 nm, more than 50 nm, more than 100 nm, more than 500 nm, more than 1 um, more than 5um, more than 25 um, more than 40 um. The thickness of the mesh may be less than 50 um, less than 35 um, less than 10 um.

The material of at least part of the conductive film of this disclosure may be graphene.

Alternatively, the material of the mesh 101 may be non-metallic. For example, the material of the mesh may be graphene. A thickness of graphene mesh may be in the range of 0.1 nm-50 um. Specifically, the thickness of metal mesh may be more than 0.1 nm, 1 nm, 5 nm, 10 nm, more than 50 nm, more than 100 nm, more than 500 nm, more than 1 um, more than 5um, more than 25 um, more than 40 um. The thickness of the mesh may be less than 50 um, less than 35 um, less than 10 um.

Alternatively, at least part of the conductive film of this disclosure may be a conductive adhesive. Alternatively, the material of at least part of the conductive film of this disclosure may be polymeric or biobased material comprising conductive fillers. Specifically, the material of at least part of the conductive film may be nano-fibrillated or micro-fibrillated cellulose comprising a conductive filler. The conductive filler may be carbon, or carbon fibre, or carbon nanotubes, or metallic particle, or similar.

A thickness of the conductive film may be in the range of 0.1-300 um. A thickness of the conductive adhesive may be more than 0.1 um, more than 1 um, more than 5 um, more than 20 um, more than 50 um, more than 100 um, more than 250 um. A thickness of the conductive adhesive may be less than 300 um, less than 150 um, less than 80 um, less than 40 um, less than 10 um, less than 2 um, less than 0.5 um.

The fabrication process of the mesh 101 may vary depending on the material. The mesh 101 may be fabricated by forming the openings in the thin film of material using e.g. microfabrication, laser ablation of other similar processes. The mesh 101 may be formed by depositing the ink with suitable electrical properties using e.g. inkjet printing or stenciling. Another alternative way of mesh 101 fabrication is by weaving the conductive wires or fibres.

### Electrical Properties of the Skin Interface Structure

The conductivity of the skin interface structure may be greater than the conductivity of the outermost surface of the electrode structure.

The skin interface structure 100 may be configured with specific electrical properties in order enhance the quality of the signal passing from the skin to the electrode structure. A conductivity of the skin interface structure 100 may be greater than the conductivity of the outermost surface of the electrode structure in contact with the skin. The conductivity of the skin interface structure may be in the range of conductivities of the electrically conductive metals. For example, the conductivity of the skin interface structure may be 1×10⁷-6.3×10⁷ S/m. The conductivity of the adhesive layer of the electrode structure may be between 0.001 and 100 S/m. Alternatively, a resistivity of the skin interface structure may be smaller than the resistance of the outermost surface of the electrode structure in contact with the skin.

### Skin Interface Structure Embodiments

The skin interface structure 100 may additionally comprise a supporting structure 301. The supporting structure may be electrically conductive. The supporting structure may be electrically non-conductive. The supporting structure may be needed if the thickness of the mesh is so small that the mesh is unable to retain its shape without additional support. Thus, the supporting structure may be configured to reinforce the conductive mesh. The exemplary skin interface structures with and without the supporting structure 301 are illustrated in FIG. 3a:
a) The skin interface structure 100 with the rectangular mesh structure 101 comprising rectangular openings 102 integrated into a supporting structure 301;
b) The skin interface structure 100 with the rectangular mesh structure 101 comprising rectangular openings 102, and further comprising the supporting structure 301 integrated into it;
c) The skin interface structure 100 with rectangular mesh structure 101 comprising circular openings 102, and further comprising the supporting structure 301 integrated into it;
d) The skin interface structure 100 with the circular mesh structure 101 comprising circular openings 102, and comprising no supporting structure;
e) The skin interface structure 100 with alternative circular mesh structure 101 wherein mesh portions extend from a nominal centre of the interface structure towards its edge. The skin interface structure 100 comprises no supporting structure in this case;
f) The skin interface structure 100 with alternative rectangular mesh structure 101 wherein mesh portions extend between opposite edges of the interface structure is a zigzag shape. The skin interface structure 100 comprises no supporting structure in this case.

The supporting structure 301 and the mesh 101 may be connected during manufacturing process. For example, more rigid fibres of the supporting structure may be added to the electrically conductive fibres that need support during weaving process.

Other designs of the skin interface structure comprising the electrically conductive mesh and additional electrically conductive or electrically non-conductive supporting structures may be developed. The described examples should not be viewed in a limiting sense.

### Electrode Structure With the Skin Interface Structure

The skin interface structure is embedded into the outermost surface of the electrode structure.

The skin interface structure 100 of this disclosure may be used with different types of the electrode structures. The skin interface structure 100 may be used with a commercially available electrode structures as was described above. The commercially available electrode structures may be, for example, ECG or EMG electrode as in FIG. 1 c). The skin interface structure may be for a disposable electrode structure. The skin interface structure may be for a reusable electrode structure. The skin interface structure may be for a wet electrode structure. The skin interface structure may be for a dry electrode structure. The electrode structure with the skin interface structure may also be referred to as the bioelectrode.

The skin interface structure 100 may be directly attached to the outermost surface 207 of the electrode structure 200. In other words, the skin interface structure 100 may be attached to the surface of the electrode structure 200, which is configured to be attached to the skin if used without the skin interface structure. In particular, the skin interface structure 100 may be the only structure between the outermost surface of the electrode structures and the skin. In other words, the top surface of the skin interface structure 100 may be attached to the outermost surface of the electrode structures, and the bottom surface of the skin interface structure 100 may be configured to attach to the skin.

The skin interface structure 100 may be inseparably integrated into the electrode structure 200. The skin interface structure 100 may also be embedded into the outermost surface 207 of the electrode structure 200. In other words, the skin interface structure 100 may be attached to the outermost surface 207 during the electrode structure fabrication process by, for example, bonding, deposition, printing and so on. Thus, the skin interface structure 100 may be disposed together with the disposable electrode structure after use. Alternatively, the skin interface structure 100 may be sterilized together with the reusable electrode structure after use. The skin interface structure 100 may be configured to be non-separable from the electrode structure 200.

The skin interface structure 100 may cover the entire outermost surface 207 of the electrode structure 200. In other words, the skin interface structure 100 may extend across the approximately entire outermost surface 207 of the electrode structure 200 as illustrated in FIG. 1 b). Alternatively, the skin interface structure 100 may cover part of the outermost surface 207 of the electrode structure 200 as illustrated in FIG. 1 c). Specifically, the centre of the skin interface structure 100 may be approximately aligned with the centre of the bottom surface of the carrier of the electrode structure 200 and partly extend towards the edges of the outermost surface 207. The centre of the skin interface structure 100 may alternatively be approximately aligned with the centre of the conductive element of the electrode structure 200 and extend away towards the edges of the outermost surface 207.

If the outermost surface 207 is entirely formed by the adhesive layer 205, the skin interface structure 100 may be on the adhesive layer 205 of the electrode structure. In that case, the adhesive layer 205 may extend around the skin interface structure 100. The adhesive layer 205 may be configured to adhere to the skin around the skin interface structure 100. The adhesive layer 205 may be configured to adhere to the skin through at least one opening in the skin interface structure 100.

The skin interface structure 100 may alternatively be a separate product. The skin interface structure may comprise an electrically conductive thin film with at least one opening. The skin interface structure may be configured to attach to the outermost surface of the electrode structure before attachment of the electrode structure to the skin. The skin interface structure may be reused with other electrode structures. For example, medical personnel may clean and sterilize the skin interface structure prior to each use and manually place it between the electrode structure and the skin. Alternatively, the skin interface structure may be disposed after use together with the disposable electrode structure.

### An Example of the Electrode Structure With the Skin Interface Structure

An example of the electrode structure 200 with the skin interface structure 100 is illustrated in FIG. 3b. On the left of FIG. 3b, the electrode structure with the skin interface structure is shown from the bottom side which is configured to be attached to the skin. On the right of the FIG. 3b, the top side of the electrode structure with the skin interface structure is shown.

The carrier 201 of the electrode structure 200 of this embodiment may have openings. In particular, the openings of the carrier 201 may have hexagonal shape. The carrier 201 may have 15 openings in this example. The openings in the carrier 201 may be patterned using laser micromachining, die cutting, punching, or similar. The diameter of the carrier 201 may be 30-50 mm. In particular, the diameter of the carrier 201 may be 45 mm. The thickness of the carrier 201 may be 300-700 um. In particular, the thickness of the carrier 201 may be 500 um. The material of the carrier 201 may be foam. The material of the carrier 201 may be, for example, Avery Dennison MED 5699R foam.

The carrier adhesive 208 of the electrode structure 200 of this embodiment may have openings. In particular, the openings of the carrier adhesive 208 may have round shape. The carrier adhesive 208 may have 500 openings in this example. The openings in the carrier adhesive 208 may be perforated using laser micromachining. The material of the carrier adhesive 208 may be nonwoven. The material of the carrier adhesive 208 may particularly be polyester tape 3M 4076. The diameter of the carrier adhesive 208 may be 30-50 mm. In particular, the diameter of the carrier adhesive 208 may be 45 mm. The thickness of the carrier adhesive 208 may be 30-500 um. In particular, the thickness of the carrier adhesive 208 may be 250 um.

The material of the conductive element 206 (snap button 203 of the conductive element 206 is illustrated in the right image of FIG. 3b) of this example may be stainless steel. Alternatively, the material of the conductive element of this example may be silver chloride (Ag/Cl), or conductive polymer. The material of the conductive layer 204 of this example may be stainless steel. Alternatively, the material of the conductive layer 204 of this example may be silver chloride (Ag/Cl), or conductive polymer film.

The skin interface structure 100 of this embodiment may comprise an electrically conductive film with 500 openings in it. The shape of the openings in the skin interface structure 100 may be round. The material of the skin interface structure 100 may be, for example, Flexcon Omni-Wave H-502 tape. The openings in the skin interface structure 100 may be perforated using laser micromachining. The diameter of the skin interface structure 100 may be 20-50 mm. In particular, the diameter of the skin interface structure 100 may be 35 mm. The thickness of the skin interface structure 100 may be 10-200 um. In particular, the thickness of the skin interface structure 100 may be 50 um.

In this embodiment, the skin interface structure 100 may be attached to the outermost surface of the electrode structure. In particular, the skin interface structure 100 may cover part of the outermost surface 207 of the electrode structure 200. In FIG. 3b, the skin interface structure is visible as a grey circle in the image on the left. Specifically, the skin interface structure 100 may be attached to both the carrier adhesive 208 and the conductive layer 204. Due to thickness of the skin interface structure 100, it may flex in order to be attached to both the carrier adhesive 208 and the conductive layer 204.

### An Example of the Bioelectrode With the Liner Film

The electrode structure 200 with the skin interface structure 100 of this disclosure may optionally comprise a liner film 209 (visible in FIG. 3b) attached at least to the skin interface structure 100. Alternatively, the liner film may be attached to the skin interface structure 100 and to the outermost surface 207 of the electrode structure 200. The liner film may be configured to be removed from the skin interface structure 100 prior to attaching the electrode structure 200 with the skin interface structure 100 to the skin. Alternatively, the liner film may be configured to be removed from the skin interface structure 100 and the outermost surface 207 of the electrode structure 200 prior to attaching the electrode structure 200 with the skin interface structure 100 to the skin. The diameter of the liner film 209 may be 30-70 mm. In particular, the diameter of the liner film 209 may be 50 mm. The thickness of the liner film 209 may be 50-300 um. In particular, the thickness of the liner film 209 may be 125 um.

### An Example of the Bioelectrode With the Skin Caring Layer

The bioelectrode of this disclosure may optionally comprise a skin caring layer 210, as illustrated in FIGS. 2d and 2e. The adhesive layer 205 may comprise the skin caring layer as in FIG. 2d. In other words, the skin caring layer may be embedded into the adhesive layer 205 of the electrode structure 200. Alternatively, the skin caring layer 210 may be attached to the carrier adhesive 208.

Alternatively, the skin caring layer 210 may be part of the skin interface structure 100 as in FIG. 2e. In other words, the skin caring layer 210 may be attached to the electrically conductive film of the skin interface structure 100. In particular, the electrically conductive film (the mesh) may comprise a top surface, and the skin caring layer 210 may be an additional layer attached to the top surface of the electrically conductive film (the mesh). Alternatively, the skin caring layer 210 may be at the top surface of at least part of the skin interface structure. Alternatively, the skin caring layer 210 may be at the bottom surface of at least part of the skin interface structure. For example, the skin caring layer 210 may be at the supporting structure 301 of the skin interface structure. The skin caring layer 210 may be arranged so that it is in between the electrode structure 200 and the electrically conductive film of the skin interface structure 100 when attached to the skin.

The at least one material component of the skin caring layer may be aloe vera. The at least one material component of the skin caring layer may be a medicament. Specifically, the at least one material component of the skin caring layer may be selected from the not exclusive list of: vitamin A, vitamin B complex, vitamin C, vitamin D, vitamin E, vitamin K, zinc, copper, iron, selenium, beta-carotene, collagen, I-arginine, calcium, magnesium, bromelain, aspirin, ibuprofen, naproxen, diclofenac, celecoxib, cortisone. Alternatively, the material of the skin caring structure may comprise more than one material component. Specifically, the material of the skin caring structure may comprise combination of material components such as the ones mentioned above. Alternatively, the material of the skin caring layer may comprise more than one material component.

The skin caring layer may comprise a first film and a second film attached to each other, wherein the second film may be formed from the at least one material component described above. For example, the first film may be a thin film, which may be dipped into a liquid solution comprising at least one material component described above, so that a coating of the second film is formed on at least one surface the first film. Alternatively, the liquid solution comprising at least one material component described above may be deposited to the first film with other methods, such as spray coating. Alternatively, the skin caring layer may comprise only one film, which may be formed from the liquid solution comprising at least one material component described above. In this case, for example, the at least one surface of the conductive film of the skin interface structure may, for example, be coated with the skin caring layer.

The purpose of the skin caring layer may be to reduce skin irritation, inflammation, or to nourish the skin, or to heal the skin when the bioelectrode is attached to the skin.

### ECG Signal Quality Improvement

The use of the skin interface structure 100 of this disclosure with the disposable ECG electrode structure leads to an improved raw ECG signal quality. The assessment of the signal quality may be conducted according to the exemplary protocol:
(1) (Optional) Removing of excess body hair with, for example, a razor blade;
(2) Wiping the skin with rubbing alcohol;
(3) (Optional) Removing of the dead skin cells with an abrader, for example, an abrader tape;
(4) Attaching the ECG electrodes and the electrode with the skin interface structure to the cleaned and optionally rubbed area of the skin;
(5) Connecting wires from the measurement device to the electrodes;
(6) Performing ECG of impedance measurements;

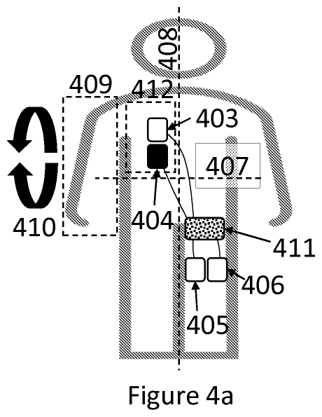

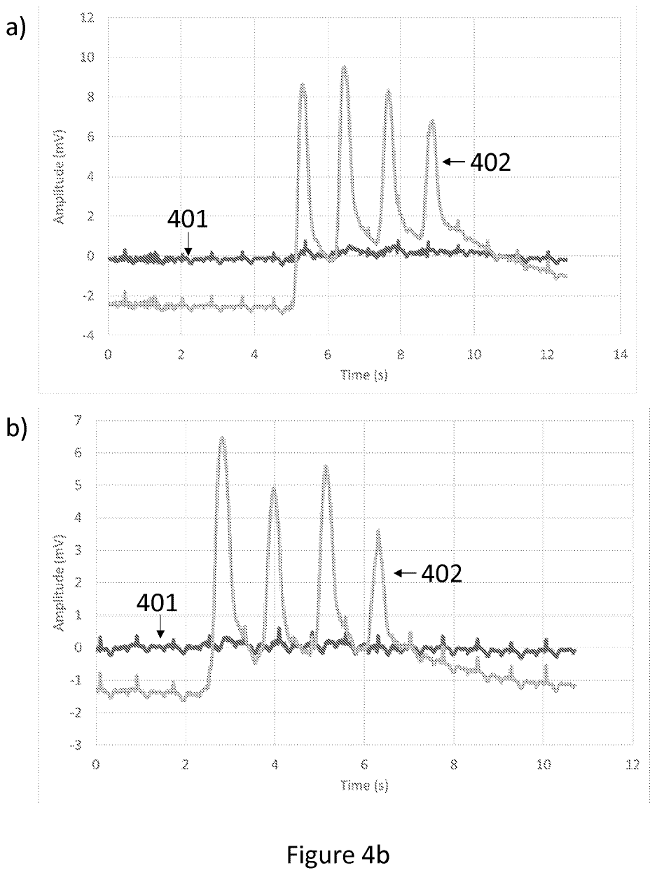

For assessment of the functionality of the electrodes, the measurement may be arranged as illustrated in FIG. 4a. For the measurements, the patient may be seated still. The lines 407-408 may define a right upper abdominal quadrant 412 of the body. A right arm 409 of the patient may move in a direction 410 during the measurement and may partly expose the right upper quadrant 412 to the same motion. The ECG electrodes may be attached to the skin of the patient as follows:
(1) A first electrode 403 and a second electrode 404 may be attached to the skin in the right upper quadrant 412; The first electrode 403 may correspond to the electrode structure 200, and the second electrode 404 may correspond to the electrode structure 200 with the skin interface structure 100.
(2) The electrodes 403-404 may be connected to a shimmer 411 which registers the electrical signals from the electrodes.
(3) A reference electrode 406 may be at the left thigh of the patient which experiences a negligible motion during right arm rotation. The reference electrode 406 may be used for common mode rejection.
(4) A bi-polar electrode 405 may be at the left thigh of the patient near the reference electrode 406. Both bi-polar and reference electrodes 405-406 may be connected to the shimmer 411. Both bi-polar and reference electrodes 405-406 may correspond to the electrode structure 200.

The differential voltage measurement may be performed during rotation of the right arm by the patient. The voltage between the first electrode 403 and the bi-polar electrode 405, and the second electrode 404 and the bi-polar electrode 405 may be measured. The output of the measurements is presented in FIG. 4b.

The data in FIG. 4b illustrates a) the raw signal from one channel ECG-measurement during the arm movement from the electrode structure with the skin interface structure (401) and without the skin interface structure (402) 30 minutes after the placement of the electrode structure and the skin interface structure, and b) the raw signal during the arm movement from the electrode structure with the skin interface structure (401) and without the skin interface structure (402) 1 hour after the first measurement. The data demonstrates that the skin interface structure between the electrode structure and the skin may reduce motion artefacts and improve quality of the raw ECG data.

1.-15. (canceled)

16. A bioelectrode comprising:
an electrode structure comprising
a carrier having a top surface and a bottom surface,
a conductive element extending through the carrier to the bottom surface, and
an adhesive layer which at least partly covers the bottom surface of the carrier and forms an outermost surface of the electrode structure;
wherein the bioelectrode further comprises:
a skin interface structure on the outermost surface of the electrode structure, the skin interface structure comprising an electrically conductive film having at least one opening in it.

17. The bioelectrode according to claim 16, wherein the conductive film comprises metal.

18. The bioelectrode according to claim 17, wherein a thickness of the conductive film is 10nm-50 um.

19. The bioelectrode according to claim 16, wherein the conductive film comprises graphene.

20. The bioelectrode according to claim 16, wherein the conductive film comprises carbon fibre.

21. The bioelectrode according to claim 16, wherein the adhesive layer of the electrode structure is electrically conductive.

22. The bioelectrode according to claim 16, wherein the skin interface structure is embedded into the outermost surface of the electrode structure.

23. The bioelectrode according to claim 16, wherein the conductivity of the skin interface structure is greater than the conductivity of the outermost surface of the electrode structure.

24. The bioelectrode according to claim 16, wherein the carrier of the electrode structure comprises at least one opening in it.

25. The bioelectrode according to claim 16, wherein the adhesive layer of the electrode structure comprises at least one opening in it.

26. The bioelectrode according to claim 16, wherein the bioelectrode further comprises a skin caring layer.

27. The bioelectrode according to claim 26, wherein the electrically conductive film of the skin interface structure comprises a top surface, and wherein the skin caring layer is at the top surface of the electrically conductive film.

28. The bioelectrode according to claim 26, wherein the adhesive layer of the electrode structure comprises the skin caring layer.

29. The bioelectrode according to claim 26, wherein at least one material of the skin caring layer is aloe vera.

30. The bioelectrode according to claim 26, wherein at least one material of the skin caring layer is a medicament.
